(19) United States
(12) Patent Application Publication
(10) Pub. No.: US 2026/0245706 A1
(43) Pub. Date: Aug. 20, 2026

(54) Real-Time Job Distribution and Nurse Tracking Platform

(71) Applicant: Gavaskar Sharp, Duluth, GA (US)

(72) Inventor: Gavaskar Sharp, Duluth, GA (US)

(21) Appl. No.: 19/058,101

(22) Filed: Feb. 20, 2025

(51) Int. Cl. G16H 40/20; G06Q 10/0631; G16H 80/00; H04W 4/021

(52) U.S. Cl. CPC G16H 40/20; G06Q 10/063112; G06Q 10/063114; G16H 80/00; H04W 4/021

(57) ABSTRACT

The present invention, NURSEPRO, is a real-time job distribution and nurse tracking platform designed to enhance workforce management in the healthcare industry. The system enables healthcare providers to schedule, assign, and monitor nursing tasks while leveraging geolocation technology, automated scheduling, and real-time tracking to reduce nurse no-shows and improve operational efficiency. The platform comprises a web-based and mobile application interface, a job scheduling engine that dynamically assigns nurses based on predefined criteria, a real-time tracking module utilizing GPS and geofencing for location monitoring, an automated alert system for notifications regarding job status and deviations, and a reporting and analytics module that provides workforce performance insights. The invention ensures enhanced accountability, optimized resource allocation, and improved patient care outcomes by offering a seamless solution for real-time nurse tracking and scheduling management.

### BACKGROUND OF THE INVENTION

### Field of Invention

The present invention relates to systems and methods for workforce management in the healthcare industry. More specifically, the invention pertains to a real-time job distribution and nurse tracking platform that enables healthcare providers to schedule, monitor, and optimize nurse assignments across multiple locations. The invention leverages geolocation technology, scheduling algorithms, and real-time tracking to mitigate nurse no-shows and enhance accountability in outpatient and clinical care settings.

### BRIEF SUMMARY OF THE INVENTION

The present invention, NURSEPRO, is a real-time job distribution and nurse tracking platform designed to streamline workforce management in the healthcare industry. The system allows healthcare providers to schedule nurses for daily, weekly, or monthly assignments while enabling real-time tracking of nurse locations to ensure scheduled tasks are completed. By integrating geolocation technology, automated scheduling, and real-time monitoring, the invention addresses the persistent issue of nurse no-shows, improving operational efficiency and patient care outcomes. The platform provides enhanced accountability, allowing administrators to track nurse movements between assignments and optimize resource allocation.

### BRIEF DESCRIPTION OF THE FIGURES

FIG. 1: A user interface displayed on a mobile device, showing the onboarding/signup page, the nurse location tracking page, and the estimated arrival time page.

FIG. 2: The nurse onboarding process, illustrating the creation of a user account by requesting personal information and availability schedule.

### DETAILED DESCRIPTION

The present invention, NURSEPRO, is a real-time job distribution and nurse tracking platform designed to improve workforce management in the healthcare industry. By integrating geolocation technology, automated scheduling, and real-time monitoring, the platform addresses the persistent issue of nurse no-shows while ensuring efficient and accountable healthcare service delivery. The system enables healthcare providers to schedule nurses for daily, weekly, or monthly assignments while tracking their movement between locations in real time. This ensures that scheduled tasks are completed as planned, reducing disruptions in clinical and outpatient care settings.

The NURSEPRO platform comprises a web-based and mobile application interface that serves both nurses and healthcare administrators. The interface allows for seamless job scheduling, assignment tracking, and real-time location monitoring. Healthcare administrators can use the system to allocate shifts based on nurse availability, geographic proximity, and specialty qualifications, ensuring optimal resource utilization. The system also prevents nurse overbooking or underutilization by balancing workloads across multiple assignments. Through an integrated scheduling algorithm, the platform assigns jobs dynamically, optimizing workforce efficiency while reducing administrative burdens.

A core component of the invention is its real-time tracking module, which utilizes GPS and geofencing technology to monitor nurse movement between assigned locations. The platform provides continuous status updates, including estimated arrival times and real-time location sharing, ensuring that administrators have full visibility into workforce activity. The system generates automatic alerts if a nurse fails to check in at an assigned location or deviates from a planned route. These alerts allow for immediate intervention, reducing the risk of patient care delays due to unexpected nurse absences. Location-based timestamps further enhance accountability by verifying that nurses arrive and depart from assignments as scheduled.

The communication system within NURSEPRO enhances operational efficiency by automating notifications and alerts. Nurses receive push notifications or SMS alerts regarding upcoming assignments, schedule changes, or urgent job requests. Healthcare administrators can send real-time updates or emergency notifications to ensure seamless coordination across teams. This feature reduces miscommunication and enhances the responsiveness of nursing staff to changing healthcare demands. Additionally, the platform incorporates a reporting and analytics module that provides data-driven insights into workforce performance, including nurse punctuality, job completion rates, and overall efficiency. These analytics allow administrators to identify patterns, make informed scheduling decisions, and optimize staffing strategies for improved patient outcomes.

By integrating all these features into a single platform, NURSEPRO creates a seamless and efficient solution for managing nurse assignments in real time. The system ensures that healthcare providers have full visibility into workforce operations while holding nurses accountable for their scheduled responsibilities. Through real-time tracking, automated scheduling, and communication enhancements, the invention significantly reduces nurse no-shows and improves the reliability of healthcare service delivery.

Below is a code representation of the core functionalities of the NURSEPRO system. This includes user onboarding, nurse tracking, job scheduling, and alert notifications, which are fundamental to the system's operation.

```
  # NursePro System: Pseudo-Code Representation
  # Database structure for users
  class Nurse:
    def _{——}init_{——}(self, name, license_id, contact_info, availability, location):
      self.name = name
      self.license_id = license_id
      self.contact_info = contact_info
      self.availability = availability
      self.location = location
      self.status = “Available” # Can be ‘Available’, ‘On Duty’, or ‘Offline’
  class Job:
    def _{——}init_{——}(self, job_id, location, assigned_nurse, status, start_time,
end_time):
      self.job_id = job_id
      self.location = location
      self.assigned_nurse = assigned_nurse
      self.status = status # ‘Pending’, ‘In Progress', ‘Completed’, ‘Missed’
      self.start_time = start_time
      self.end_time = end_time
  # Database for tracking nurses and jobs
  nurses_list = [ ]
  jobs_list = [ ]
  # Nurse onboarding process
  def nurse_onboarding(name, license_id, contact_info, availability):
    new_nurse = Nurse(name, license_id, contact_info, availability,
location=None)
    nurses_list.append(new_nurse)
    return f“Nurse {name} successfully onboarded.”
  # Job scheduling system
  def assign_nurse_to_job(job_id, location):
    available_nurses = [nurse for nurse in nurses_list if nurse.status ==
“Available”]
    if not available_nurses:
      return “No available nurses at the moment.”
    assigned_nurse = available_nurses[0] # Assign first available nurse (logic can
be optimized)
    new_job = Job(job_id, location, assigned_nurse, “Pending”, start_time=None,
end_time=None)
    jobs_list.append(new_job)
    assigned_nurse.status = “On Duty”
    return f“Nurse {assigned_nurse.name} assigned to job {job_id} at {location}.”
  # Real-time tracking of nurses
  def update_nurse_location(nurse_name, new_location):
    for nurse in nurses_list:
      if nurse.name == nurse_name:
        nurse.location = new_location
        return f“Location for Nurse {nurse_name} updated to {new_location}.”
    return “Nurse not found.”
  # ETA Calculation (Simplified)
  def estimate_arrival_time(nurse_name, job_location):
    for nurse in nurses_list:
      if nurse.name == nurse_name and nurse.location:
        distance = calculate_distance(nurse.location, job_location) # Placeholder
function
        estimated_time = distance / AVERAGE_SPEED # Assuming constant
speed
        return f“Estimated arrival time for Nurse {nurse_name}: {estimated_time}
minutes.”
    return “Nurse location not available.”
  # Alert system for nurse no-shows
  def check_no_show(nurse_name, job_id):
    for job in jobs_list:
      if job.job_id == job_id and job.assigned_nurse.name == nurse_name:
        if job.status == “Pending” and time_since_scheduled_start(job.start_time)
> NO_SHOW_THRESHOLD:
          send_alert(f“Nurse {nurse_name} has not checked in for job {job_id}.
Immediate action required.”)
          job.status = “Missed”
          return f“Alert sent: Nurse {nurse_name} marked as a no-show for job
{job_id}.”
    return “No issues detected.”
  # Communication system
  def send_alert(message):
    print(f“ALERT: {message}”) # Placeholder for push notification or SMS
integration
    return True
  # Utility function (placeholder for real distance calculation)
  def calculate_distance(start_location, end_location):
    return 10 # Placeholder distance in miles
  # Mock constants for demonstration
  AVERAGE_SPEED = 30 # mph
  NO_SHOW_THRESHOLD = 15 # minutes
  # Example usage
  print(nurse_onboarding(“Alice Johnson”, “RN12345”, “alice@example.com”,
“Weekdays 8 AM - 5 PM”))
  print(assign_nurse_to_job(“JOB001”, “Hospital A”))
  print(update_nurse_location(“Alice Johnson”, “Downtown Clinic”))
  print(estimate_arrival_time(“Alice Johnson”, “Hospital A”))
  print(check_no_show(“Alice Johnson”, “JOB001”))
```

### DETAILED DESCRIPTION OF FIGURES

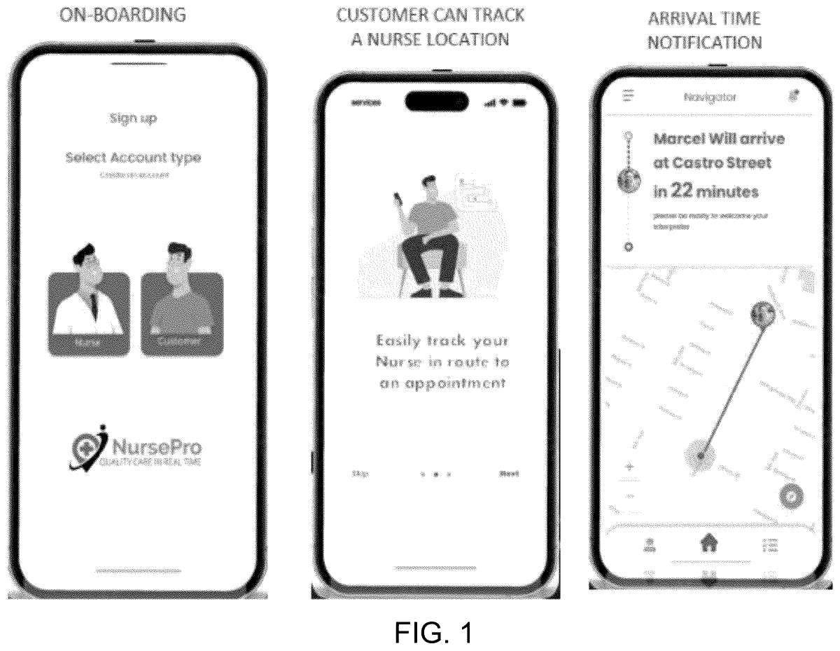

FIG. 1: illustrates the user interface of the NURSEPRO mobile application as displayed on a mobile device. The figure comprises three primary screens: (1) the onboarding/signup page, where users register for the platform by providing necessary credentials and authorization; (2) the nurse location tracking page, which allows users to monitor the real-time location of an assigned nurse as they travel to a designated job site using GPS-based tracking; and (3) the arrival time page, which displays an estimated time of arrival (ETA) based on the nurse's real-time movement and traffic conditions. These interface components work together to streamline user onboarding, provide real-time workforce visibility, and enhance the overall scheduling efficiency of healthcare personnel.

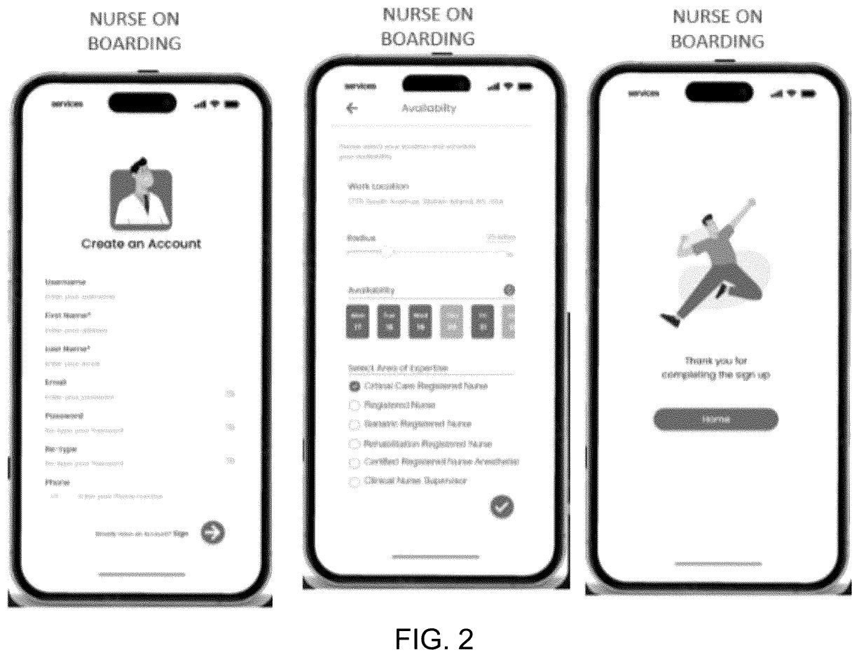

FIG. 2: depicts the nurse onboarding process, which facilitates the creation of a nurse account within the NURSEPRO platform. The process begins by requesting essential personal information, such as full name, contact details, licensing credentials, and employment history. The system then prompts the nurse to set availability preferences using an interactive scheduler, allowing them to specify preferred working days, shift durations, and geographic service areas. This onboarding process ensures that nurses are properly authenticated and that their scheduling preferences are integrated into the system's job scheduling engine, optimizing job assignments based on availability and qualifications.

What is claimed is:

1. A real-time nurse tracking and job distribution system, comprising:
a. a web-based and mobile application interface configured to enable healthcare administrators to schedule, assign, and monitor nurse jobs in real time;
b. a job scheduling engine that dynamically assigns nursing tasks based on predefined criteria including nurse availability, geographic proximity, specialty qualifications, and workload balancing;
c. a real-time tracking module utilizing GPS and geofencing technology to monitor the location and movement of nurses between assigned job locations;
d. n automated alert system that generates notifications for schedule changes, estimated arrival times, and deviations from assigned job locations; and
e. a reporting and analytics module that collects and processes workforce performance data, including nurse punctuality, job completion rates, and operational efficiency.

2. The system of claim 1, wherein the real-time tracking module further comprises a geofencing system that creates virtual geographic boundaries around job locations to trigger automated check-in and check-out verifications.

3. The system of claim 1, wherein the job scheduling engine optimizes assignments using machine learning algorithms to predict nurse availability, optimize job distribution, and reduce scheduling conflicts.

4. The system of claim 1, wherein the automated alert system sends real-time push notifications, SMS messages, and email alerts to both nurses and administrators regarding job status updates, delays, or urgent rescheduling needs.

5. The system of claim 1, wherein the web-based and mobile application interface enables nurses to view, accept, decline, or request modifications to assigned shifts in real time.

6. The system of claim 1, wherein the real-time tracking module records and stores timestamped location data for each nurse, ensuring verifiable proof of arrival and departure at assigned job sites.

7. The system of claim 1, wherein the reporting and analytics module provides data visualization dashboards that display workforce performance metrics, job completion rates, and historical nurse movement patterns.

8. The system of claim 1, wherein the geofencing system automatically triggers an alert if a nurse fails to arrive at a designated job site within a predefined time threshold.

9. The system of claim 1, wherein the job scheduling engine integrates with third-party healthcare management systems and electronic health records (EHRs) to streamline administrative workflows.

10. The system of claim 1, wherein the real-time tracking module allows patients or healthcare facilities to view the estimated arrival time of an assigned nurse for improved patient coordination.

11. The system of claim 1, further comprising a secure cloud-based storage system that maintains historical records of nurse schedules, tracking data, and job performance metrics for audit and compliance purposes.

12. The system of claim 1, wherein the automated alert system is configured to escalate notifications to supervisory personnel if a nurse fails to respond to an assignment alert within a predefined period.

13. The system of claim 1, wherein the real-time tracking module includes a mobile device compatibility feature, enabling nurses to use smartphones, tablets, or wearable devices to update job status and location data.

14. The system of claim 1, wherein the job scheduling engine prioritizes assignments based on urgency level, patient condition, and nurse skill set to ensure appropriate nurse-to-patient matching.

15. The system of claim 1, wherein the communication system enables two-way messaging between nurses and administrators, allowing for real-time coordination and issue resolution.
